(19) United States
(12) Patent Application Publication
(10) Pub. No.: US 2026/0311832 A1
(43) Pub. Date: Oct. 8, 2026

(54) METHOD AND SYSTEM FOR CONTEXT-AWARE RADIOLOGY REPORT GENERATION USING A JSON-BASED ENGINE AND LARGE LANGUAGE MODELS

(71) Applicant: Radpair Corp., Knoxville, TN (US)

(72) Inventor: Avez Rizvi, Knoxville, TN (US)

(21) Appl. No.: 19/641,167

(22) Filed: Apr. 7, 2026

Related U.S. Application Data
(60) Provisional application No. 63/784,904, filed on Apr. 7, 2025.

(51) Int. Cl. G16H 15/00; G06F 40/279

(52) U.S. Cl. CPC G16H 15/00; G06F 40/279

(57) ABSTRACT

The present technology relates to methods and systems for context-aware report generation using a JSON-based engine and large language models, particularly for use in the practice of radiology. According to certain embodiments, the method comprises: receiving a dictation; selecting a JSON template; extracting one or more keywords from the dictation; wherein, when at least one keyword is identified, populating at least one section of the JSON template based on the at least one keyword, and when no keyword is identified, retaining a default version of the JSON template; assembling a dynamic prompt based on the JSON template and any populated section; and generating a report based on the dynamic prompt and the dictation.

### CROSS-REFERENCE TO RELATED APPLICATIONS

This application claims the benefit of U.S. Provisional Application No. 63/784,904 filed Apr. 7, 2025, which is hereby incorporated by reference in its entirety.

### BACKGROUND

The present technology relates to methods and systems for context-aware report generation using a JavaScript Object Notation (“JSON”)-based engine and large language models (“LLM”), particularly for use in the practice of radiology.

Early innovations in radiology reporting introduced template-driven or rule-based engines to enforce structured content, for example, checklist-style reporting systems. In a checklist-style reporting system there is a list of organs/structures presented with default normal text, and the radiologist can dictate overrides for abnormalities; at report finalization, each item becomes a section heading populated either by the dictated comment or a default statement, yielding an itemized structured report. This rule-based approach ensures consistent section organization (each relevant anatomy addressed) without relying on free-form text. Another example is using predefined report “skeletons” that auto-insert context-appropriate phrases and placeholders for specific findings, essentially a decision engine selecting template text based on exam context (e.g., different wording for referring physicians vs patients). These examples illustrate the use of predefined JSON/XML-like data structures or rules to drive report content. In fact, modern standards started adopting JSON for radiology data: DI-COM's Supplement 219 defines a JSON representation of Structured Reports to ease AI integration. This standard maps imaging results into a JSON structure to allow algorithmic post-processing. The existence of these template and rules engines (often configured via structured files) shows the use of structured data formats (XML/JSON) and decision logic to generate radiology report text in a consistent, sectioned format. However, these template and rules engines generally rely on fixed templates or simple if/then rules—lacking the dynamic language generation capabilities of modern AI.

### Extraction of Key Terms From a Radiology Lexicon for Structuring

Radiology has benefited from standard lexicons like RadLex, a comprehensive ontology of radiology terms established by the Radiological Society of North America (RSNA). Though not an exhaustive (or necessarily complete) list of medical radiology lexicon, RadLex approximates the various terms commonly used in medical radiology parlance. Current technology has leveraged such lexicons to standardize and structure report content. For instance, a hybrid reporting platform can combine structured templates with natural language processing (“NLP”): free-text portions of a report are automatically analyzed and coded with RadLex terms. The platform inserts RadLex-coded findings (with modifiers like “negated” or “affirmed”) into the structured report, and radiologists can confirm or adjust these codes before finalizing. This can be an approach to extract key terms from narrative text and map them to a lexicon for structured data capture. In a similar vein, a tool can convert dictated free text into a structured report template using NLP tied to RadLex nomenclature. As the radiologist speaks, the tool identifies entities (e.g., stone size and location in a urolithiasis CT report) and fills the appropriate fields in a template using standardized terms. These efforts underscore the use of a radiology lexicon database to ensure consistent terminology and to populate structured fields. Another example is an AI-driven solution that incorporates ontology knowledge; an impression generator associates words with concepts in an ontology database to improve its model's training and output. These examples demonstrate using controlled vocabularies (RadLex) for both data entry and post-processing of free text, thereby structuring reports. However, there is still a need for methods that dynamically generate JSON prompts or decision trees, rather than simply coding finished text.

### LLMs for Generating Radiology Reports From Structured Prompts

Early machine learning approaches (pre-LLM) take the structured findings input and the radiologist's ID/style as context, then produces a suggested impression text inserted into the report or rely on text generation from structured inputs (image-derived measurements or finding lists). The early approaches employ one or more trained language models (fine-tuned on radiology text) to draft the impression in the radiologist's preferred style. Alternative approaches generate narrative sentences from imaging data i.e., an AI image analysis yields key words which are then fed into a text generator model (using modified word embeddings and a semantic lexicon) to produce a natural-language sentence describing the image findings.

LLMs can be used to generate report sections from structured prompts (e.g., a prompt that specifies section headings, required fields, and formatting constraints) and, in some implementations, to emit structured outputs such as JSON objects for findings, modifiers, and recommendations. However, existing approaches typically rely on static templates, ad hoc prompt text, or models fine-tuned to a particular section layout, which can reduce portability across sites and templates and can lead to missing, misplaced, or extraneous sections when report formats change. Accordingly, there is a need for systems that programmatically construct and validate a structured, template-specific prompt on a per-case basis, rather than relying on fixed templates or generic prompting.

### Parsing Radiologist Dictation and Free Text Into Structured Sections

Handling free-form dictation and converting it into structured report elements has long been a goal in radiology informatics. Radiologists favor dictation for efficiency, yet structured reports improve consistency—bridging these has been challenging. Traditional dictation platforms and tools (e.g., those available under the tradenames Nuance PowerScribe One, Dragon Medical One, and M*Modal Fluency Direct) rely on standard speech recognition systems to transcribe a user's spoken notes to streamline the documentation process. Many of these platforms convert free-form dictation into structured report fields in real time. As the radiologist speaks, the system uses AI to populate the appropriate section or entry, highlight key phrases, and even provide decision support (e.g., flagging if a follow-up recommendation is needed).

LLMs can serve as parsers for unstructured dictation, including in multilingual settings, but existing solutions may not reliably map dictated content into the correct report sections across varying templates and local conventions. Accordingly, there is a need for systems and methods that parse free text into JSON-defined sections using an LLM under explicit, template-specific structural constraints.

### Conclusion—Novelty and Gaps

In summary, structured radiology reporting has been approached via template engines, controlled vocabularies, and, more recently, machine-learning models. Existing generation commonly relies on checklist-driven report assembly, static template selection, and lexicons such as RadLex to standardize terminology or to code narrative text after it is created. The arrival of large language models has enabled generation of report text from structured inputs and parsing of free text into structured outputs, but many implementations re-main prototype-level and can be sensitive to changes in template sectioning and local style. Crucially, there is no known system that (i) detects the sections of a selected report template, (ii) uses a radiology lexicon with context rules to construct a template-specific JSON prompt for an LLM on a per-case basis, and (iii) performs a post-generation reconciliation between lexicon terms in the dictation/transcript and the generated report to identify dropped findings and/or hallucinated terms. The present technology therefore discloses using a JSON-based decision engine to extract key terms (e.g., via RadLex), generate a structured prompt con-strained to the selected template's sections, and drive an LLM to produce a coherent radiology report organized according to those sections.

### BRIEF SUMMARY

The present technology relates to methods and systems for context-aware report generation using a JSON-based engine and large language models, particularly for use in the practice of radiology.

According to certain embodiments, the present technology discloses a method of report generation comprising: receiving a dictation; selecting a JSON template; extracting one or more keywords from the dictation; wherein, when at least one keyword is identified, populating at least one section of the JSON template based on the at least one keyword, and when no keyword is identified, retaining a default version of the JSON template; assembling a dynamic prompt based on the JSON template and any populated section; and generating a report based on the dynamic prompt and the dictation.

According to certain embodiments, the present technology discloses a system for report generation comprising: a device for receiving a dictation; a database with specialized lexicon; a processor for extracting keywords from the dictation based on the database; a decision engine for selecting a template; a processor for constructing a prompt based on the template and any keywords; and a large language model for generating a report based on the prompt. According to certain embodiments, the present technology discloses a method of report generation comprising: selecting a baseline prompt that includes placeholders corresponding to template sections; retrieving the JSON template from a template library; determining, using a lexicon, one or more keywords associated with the dictation; populating one or more of the placeholders with content derived from the JSON template and the one or more keywords; modifying the baseline prompt based on one or more rules that are triggered by detection of at least one keyword to generate a final prompt; and providing the final prompt and the dictation to a large language model (LLM) to generate a report.

### BRIEF DESCRIPTION OF THE DRAWINGS

FIG. 1 shows a flowchart according to an embodiment of the present technology.

FIG. 2 shows a flowchart according to an embodiment of the present technology.

FIG. 3 shows a JSON template structure according to an embodiment of the present technology.

FIG. 4 shows a sample user interface according to an embodiment of the present technology.

### DETAILED DESCRIPTION

As used herein, all singular terms refer to both singular and plural values. That is, “a” or “an” or “the” all mean “one or more.” The term “or” as used herein means any one or more of the alternatives, including all of the alternatives.

Throughout the present disclosure, when described in sequential words (for example, using “then” or “next”), such description is not limiting to the described steps in the particular order set forth, but also includes embodiments wherein the steps are presented in any order.

As used herein, “radiology” means any imaging, including but not limited to industrial imaging, medical imaging, veterinary imaging, or geological imaging.

As used herein, “in real time” means simultaneously or within up to 1 minute of simultaneously.

As used herein, “substantially” means within 5% of a value, or as understood by one of ordinary skill in the art.

Generally, in radiology, numerous radiological procedures can be employed; including computerized tomography (CT or CAT) scan; magnetic resonance imaging (MRI), X-ray, ultrasound (US) and the like. The machinery employed in such procedures is “imaging apparatus” for the purposes of the present disclosure.

As used herein, “radiologist” means a user that specializes in radiology. In certain embodiments herein, the review of images can be performed by a radiologist but can also be performed by any user who is qualified to do so. Throughout the present disclosure, when referring to a radiologist, the embodiments herein contemplate any user who is qualified and permitted to perform the task of a radiologist in connection with obtaining images.

As used herein, “user” means anyone who can access a method or system herein in order to obtain data, including images or any other information, whether a human (for example, any healthcare professional or a patient or someone authorized by the patient), a machine, AI, or a computer.

As used herein, “radiology workflow” means any system or method in the field of radiology that involves scanning of a target and then using the information obtained to diagnose a condition, including the steps included in such a system or method.

As used herein, “free-form” means any language having a source that constitutes the combination of techniques chosen by a user in telling a particular story, and that is communicated with a natural structure and style.

As used herein, “artificial intelligence” or “AI” means the simulation of human intelligence processes by machines, in particular, computer systems, including the ability to learn, read, write, create, and analyze. As used herein, “generative AI” means AI that is capable of generating text, images, or other data using generative models, which learn the pat-terns and structure of their input training data and then generate new data that has similar characteristics. As used herein, “large language models” or “LLMs” are a type of generative AI that can be multi-modal (as used in images, text, or video).

As used herein, “report” means a document that communicates findings of a user. The report can go beyond simple summarization or dictation and can involve the sorting of different categories of information including but not limited to background, analysis, findings, and conclusions, into predetermined fields within a report. The template for a report can be a prefabricated form with predetermined fields to be filled in or a blank document.

As used herein, “industry standard” means a standard that is widely accepted and adhered to within the industry, e.g., a standard for reporting that is set by the American College of Radiology, the Radiological Society of North America, the American College of Veterinary Radiology, the American Society for Nondestructive Testing, the American Society for Testing and Materials (ASTM), the Institute of Electrical and Electronics Engineers (IEEE), or similar professional organization that governs reporting standards.

Every profession uses a specialized lexicon-that is, a collection of words and their associated meanings and rules used within a specific field or domain, such as medicine or an industrial specialty. A specialized lexicon can go beyond a general dictionary by including specific terminology, definitions, and sometimes even constraints on word usage to reduce ambiguity and improve communication for a particular audience.

When using free-form dictation, syntax within phrases of a transcript created by a user will often include specialized lexicon, amongst other phrases, and normal narrative language. For example, radiologists often include radiology lexicon. Using pattern detection and referencing a continuously updated “living list” of specialized lexicon, a method herein can hold in memory any detected specialized lexicon in a free-form transcript. An LLM can convert the free-form transcript into a final report.

The present technology relates generally to automated generation of radiology reports. More specifically, it involves a JSON-based decision engine integrated with a radiology lexicon for keyword extraction and on-the-fly prompt construction for a large language model (LLM), enabling context-aware mapping of free-form dictation to structured sections in radiology reports.

JSON is a text-based data-interchange format. JSON is easy for humans to read, write, and edit while remaining machine readable. The result is a lightweight and accessible format that allows for customization based on specific needs. JSON does not support functions or many complex types, but it is well suited for template definition and customization.

Radiologists often dictate findings in a continuous, free-form manner without standardized sentence structure or grammar. Current automated solutions struggle to map these unstructured findings accurately into specific sections (e.g., “Heart,” “Lungs,” “Bones,” “Impression”) in radiology report templates.

Fine-tuning LLMs for specific radiology templates frequently leads to rigidity. If a model has been trained on a certain set of sections or style, it may insert incorrect sections or fail to adapt when the user's preferred layout changes. Retraining such fine-tuned models for each new practice or personal preference is time-consuming and expensive.

A system that dynamically identifies which sections exist in a chosen radiology template—and then uses a radiology-specific lexicon to extract key terms from dictation—would significantly improve accuracy and maintain adaptability. By constructing an on-the-fly JSON prompt for each transcript, the invention avoids overfitting to a single format and remains flexible even as templates evolve.

According to certain embodiments, the present technology is a hybrid method of automated radiology report generation that combines Radiology Lexicon Integration, a JSON-Based Decision Engine, Dynamic Prompt Construction, and a Flexible User Interface. The Radiology Lexicon Integration can be a proprietary or standardized (e.g., RadLex) terminology database containing radiology terminology (e.g., “pleural effusion,” “left basilar atelectasis”) used to detect and classify key phrases in a dictated transcript.

The JSON-based decision engine can be a schema-based structure that defines all possible sections (e.g., “Header,” and subsections under “Findings,” “Impression,” etc.). This engine serves as a rules framework mapping extracted keywords to specific sections. The JSON schema can outline the hierarchical structure of a radiology report (e.g., Header: patient information and study type; Findings: nested subsections such as “Heart,” “Lungs,” or “Lines_and_Tubes”; Impression: summaries or actionable conclusions). Each potential finding or phrase can be tagged with conditions (e.g., “contains_any_word: [‘effusion’]”). If a match is detected, an action can be triggered, such as placing the phrase into the “Pleural_Spaces” subsection.

The dynamic prompt construction can be based on the recognized sections in the template and the extracted keywords. In certain embodiments, the system constructs a custom JSON prompt per transcript and submits it to a large language model (LLM) to generate a draft radiology report in the correct section order. For example, the system can start with a baseline prompt that includes placeholders for template sections, retrieve the selected JSON template, merge it with recognized keywords from the lexicon, populate relevant sections, and then assemble a final prompt that appends or modifies specific sections based on triggered rules (e.g., “found pleural effusion”). The resulting JSON content and instructions are provided to the LLM, which produces a draft radiology report subdivided according to the JSON schema and anchored to the appropriate sections, reducing the likelihood of extraneous or missing fields.

The user interface can allow radiologists or administrators to lock or unlock sections, override default “unremarkable” phrases, and/or modify standard templates. In certain embodiments, the present technology tracks and version-controls these customizations for each user or practice. The system can be configured, for example, by toggling default phrases, locking or unlocking sections, or selecting user-specific variant templates. In certain embodiments, the user can preview how the final structured report will appear given selected toggles and custom phrases.

The present technology can be integrated with other systems. For example, the technology can integrate with external dictation software (like Dragon, Nuance, or other voice recognition services). The present technology could also be integrated into Hospital/ PACS Systems, in this integration Radiology study data, such as modality or patient demo-graphics, can be automatically fed into the JSON-based engine to help the system choose the correct template. The present technology can be integrated with APIs by exposing an API endpoint for third-party tools to submit transcripts and retrieve structured reports.

The present technology compares tokens from the dictated transcript to the lexicon using a matching algorithm. Synonyms and morphological variants (e.g., “effusions,” “atelectases”) are handled via a morphological map to recognized root terms. A set of recognized keywords or short phrases is produced, each associated with a confidence score or context indicator (e.g., negation, location).

In certain embodiments, after the LLM generates the draft report, the system performs a post-generation reconciliation between (i) specialized lexicon terms detected in the dictation/transcript and (ii) specialized lexicon terms present in the generated report. If a lexicon term detected in the dictation is absent from the generated report, the system can flag a potential dropped finding and, in some implementations, insert missing content into the appropriate section or prompt the user to confirm placement. If a lexicon term appears in the generated report but was not detected in the dictation/transcript (or otherwise supported by provided data), the system can flag a potential hallucinated term and, in some implementations, remove it, qualify it, or prompt the user for confirmation.

Unlike static, fine-tuned language models, the present technology ensures correct section mapping by ensuring that the LLM will only populate the sections actually present in the user's chosen template, preventing the insertion of extra or irrelevant sections. Further, the present technology maintains adaptability allowing revisions to templates or additional user-defined sections without requiring retraining. The JSON engine simply updates, and the LLM receives new structured instructions. Finally, the present technology offers real-time guidance by processing each transcript in real time, the system provides immediate feedback or suggests clarifications if ambiguous keywords are encountered.

According to certain embodiments, the present technology discloses a method of report generation comprising: receiving a dictation; selecting a JSON template; extracting one or more keywords from the dictation; wherein, when at least one keyword is identified, populating at least one section of the JSON template based on the at least one keyword, and when no keyword is identified, retaining a default version of the JSON template; assembling a dynamic prompt based on the JSON template and any populated section; and generating a report based on the dynamic prompt and the dictation.

In further embodiments, extracting the one or more keywords from the dictation comprises: identifying, in the dictation, one or more words that are present in a lexicon; isolating the one or more words in context within the dictation; and classifying the one or more words as the one or more keywords.

In further embodiments, generating a report comprises: submitting the dynamic prompt to a large language model (LLM); and generating, by the LLM, the report based on the dynamic prompt and the dictation.

In some embodiments, the method further comprises: performing a post-generation reconciliation between (i) the one or more keywords detected in the dictation and (ii) one or more keywords present in the generated report; wherein, when a keyword detected in the dictation is absent from the generated report, flagging a potential dropped finding; and wherein, when a keyword appears in the generated report but was not detected in the dictation, signaling a potential hallucinated term.

In certain embodiments, the method further comprises when the potential dropped finding is flagged, performing at least one of: inserting a missing keyword into an appropriate section of the report, or prompting a user to confirm placement; and when the potential hallucinated term is signaled, performing at least one of: removing the potential hallucinated term, qualifying the potential hallucinated term, or prompting the user for confirmation.

According to certain embodiments, the present technology discloses a system for report generation comprising: a device for receiving a dictation; a database with specialized lexicon; a processor for extracting keywords from the dictation based on the database; a decision engine for selecting a template; a processor for constructing a prompt based on the template and any keywords; and a large language model for generating a report based on the prompt.

According to certain embodiments, the present technology discloses a method of report generation comprising: selecting a baseline prompt that includes placeholders corresponding to template sections; retrieving the JSON template from a template library; determining, using a lexicon, one or more keywords associated with the dictation; populating one or more of the placeholders with content derived from the JSON template and the one or more keywords; modifying the baseline prompt based on one or more rules that are triggered by detection of at least one keyword to generate a final prompt; and providing the final prompt and the dictation to a large language model (LLM) to generate a report.

In certain embodiments the method further comprises: customizing the rules and JSON template for a particular user by at least one of: toggling default phrases, locking or unlocking sections, or selecting a user-specific variant template. In certain embodiments, the method further comprises: presenting a user interface that enables a user to customize the rules and JSON template.

In further embodiments, the user interface enables the user to select a report section and to at least one of (i) edit a default phrase for the report section or (ii) designate the report section as locked against modification by the LLM.

In further embodiments, the user interface enables the user to edit a rule by specifying (i) at least one keyword that triggers the rule and (ii) an action that modifies a corresponding section of the final prompt.

In certain embodiments the method further comprises: enforcing role-based permissions such that an administrator is permitted to edit the JSON template or rules and a non-administrator is restricted to selecting among pre-approved customizations.

In certain embodiments the method further comprises: storing, for each customization, at least one of a version identifier, an effective date, or an audit record identifying a user associated with the customization.

In certain embodiments the method further comprises: generating a preview report that shows how the report will appear based on the customization of the rules and JSON template.

According to one embodiment, the present disclosure could provide for Chest Radiograph Report Generation exemplified as:

User Dictation: “There is a small pleural effusion, left basilar atelectasis, and an IJ catheter in the SVC.”

Lexicon Match: “pleural effusion”→{“keyword”: “pleural effusion”, “section”: “Pleural Spaces”} “atelectasis”→{“keyword”: “basilar atelectasis”, “section”: “Lungs”} “IJ catheter”→{“keyword”: “IJ catheter”, “section”: “Lines and Tubes”}

JSON Template:
{
“Header”: {“Patient_Info”: “ . . . ”, “Study_Type”: “Chest X-ray”},
“Findings”: {
   “Pleural_Spaces”: {“phrases”: [“No effusions”]},
   “Lungs”: {“phrases”: [“No acute abnormality”]
   “Lines_and_Tubes”: {“phrases”: [“None identified”]}
},
“Impression”: {}
}

Rules: If “pleural effusion” is found, replace default phrase in “Pleural_Spaces” with “Small pleural effusion.” Insert statement about “basilar atelectasis” into “Lungs.” Insert “IJ catheter in SVC” into “Lines_and_Tubes.”

Dynamic Prompt: The system constructs a prompt for the LLM instructing it to place each recognized phrase into the corresponding section.

LLM Output: Final structured text is created, referencing “Pleural Spaces,” “Lungs,” and “Lines_and_Tubes,” and the user's default phrases are overridden by the recognized findings.

The present disclosure is adaptable, as the disclosure does not require retraining to accommodate new or altered templates. The present disclosure is precise, as the disclosure helps ensure each dictated finding is placed in the relevant section of a given template. The present disclosure allows customization, as the disclosure allows users to lock sections, override default phrases, and/or create user-specific variants of standard templates. The present disclosure can reduce error rates by minimizing the risk of the LLM adding extraneous sections or omitting essential findings, because generation is guided by a JSON schema. Finally, the present disclosure is scalable, as the JSON-based decision engine can accommodate a large array of templates and an expanding lexicon without substantially increasing model complexity.

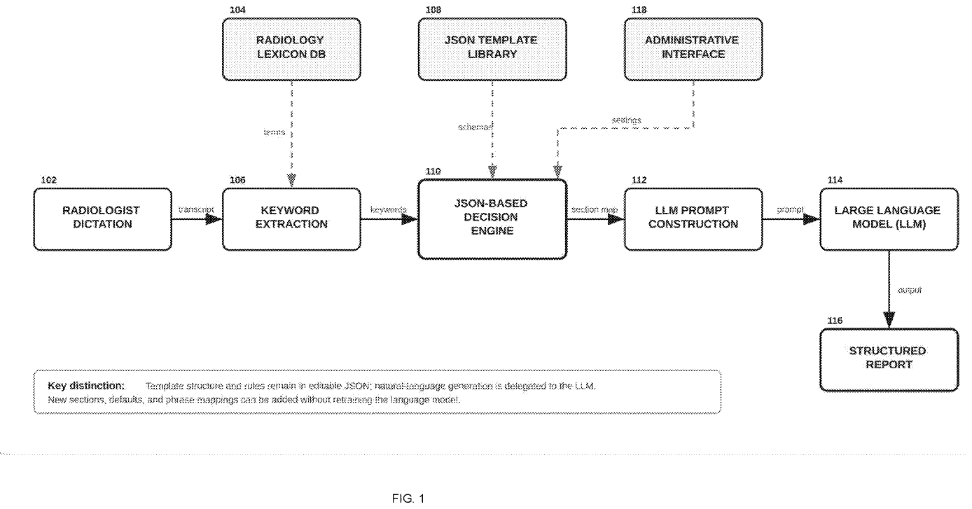

FIG. 1 shows a flowchart according to an embodiment of the present technology, specifically a system architecture overview. FIG. 1 depicts the overall architecture including input dictation 102, the radiology lexicon database 104, the JSON-based decision engine 110, and the LLM prompt construction block 112. According to certain embodiments, the present technology discloses a system for report generation comprising: a device for receiving a dictation 102; a database with specialized lexicon 104; a processor for extracting keywords 106 from the dictation based on the database 104; a decision engine 110 for selecting a template from a JSON template library 108; a processor for constructing a prompt 112 based on the template and any keywords; and a large language model 114 for generating a report 116 based on the prompt. In further embodiments, the system includes a user-facing control or administrative interface 118 for customizing the template and setting rules.

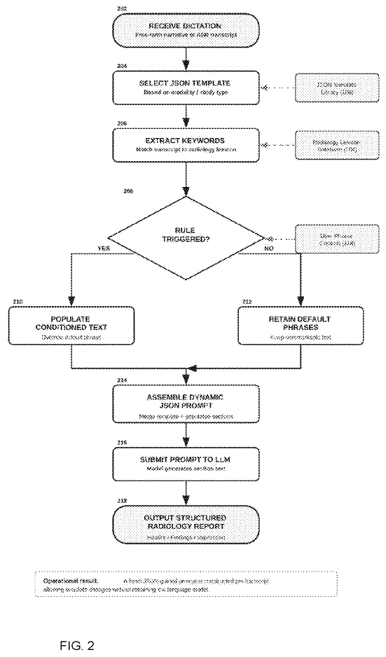

FIG. 2 shows a flowchart of a method according to one embodiment of the present technology. FIG. 2 shows a step-by-step process from dictation through keyword extraction, JSON template selection, rule application, dynamic prompt generation, and final report creation. According to certain embodiments, the present technology discloses a method of report generation comprising: receiving a dictation 202; selecting a JSON template 204 from a JSON template library 108; extracting keywords from the dictation 206 based on a lexicon database 104; wherein, when a keyword is identified, populating at least one section 210 of the JSON template based on the keyword, and when no keyword is identified, retaining the default 212 JSON template; assembling a dynamic prompt 214 based on the JSON template and any populated sections; and generating a report 218 based on the prompt, dictation, and any identified keywords. In further embodiments, generating a report comprises: submitting the prompt to an LLM 216; and generating the report using the LLM. In certain embodiments, after report generation the system performs a reconciliation step comparing specialized lexicon terms detected in the dictation/transcript against the generated report to identify potential dropped findings and/or hallucinated terms.

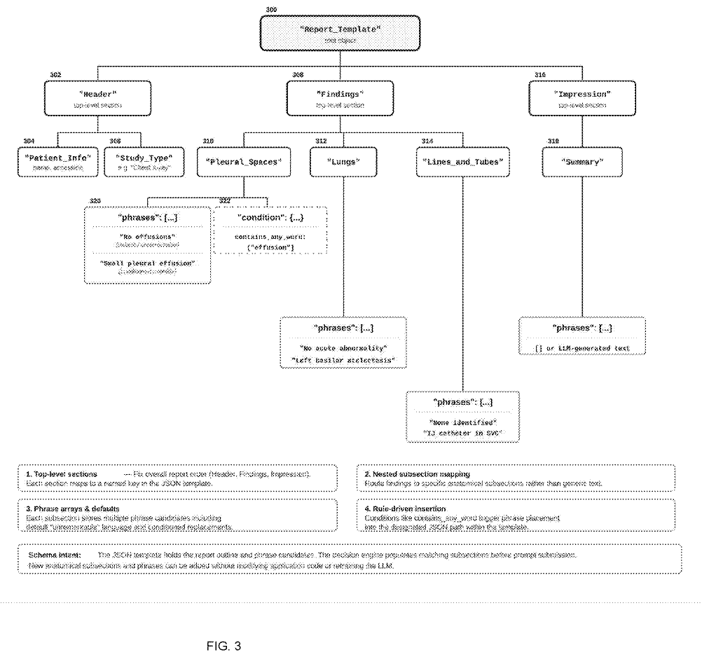

FIG. 3 is a JSON template 300 according to one embodiment. FIG. 3 illustrates a simplified JSON schema highlighting top-level sections (e.g., “Header” 302 and “Findings” 308) and nested subsections, each containing possible “Phrases” 320. The Header 302 can contain “Patient_Info” and “Study_Type” 306. The Findings section can contain subsections such as “Pleural_Spaces” 310, “Lungs” 312, or “Lines_and_Tubes” 314. In certain embodiments, these subsections can contain further elements such as “Phrases” 320 or “Condition” 322. The “Impression” 316 section can include a “Summary” 318 subsection.

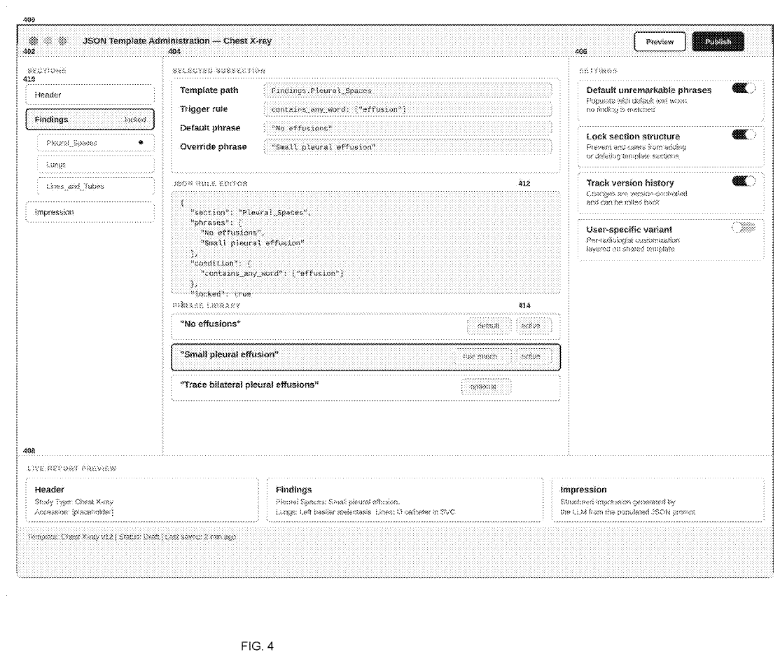

FIG. 4 shows an administrative UI window 400 for editing the JSON-based rules or sections, and toggling default “unremarkable” phrases. There can be a section menu 402, subsection menu 404, a setting menu 406, and a live report preview 408. The section menu 402 allows a user to customize sections 410 such and select a subsection to edit. The subsection 404 allows a user to edit the rules 412 that the processor will apply while generating a prompt, and to select keywords to trigger the rule in a phrase library 414. The setting menu 406 allows a user to toggle settings. The live report preview 408 allows a user to preview how the report would be generated.

Although the present technology has been described in relation to embodiments thereof, these embodiments and examples are merely exemplary and not intended to be limiting. Many other variations and modifications and other uses will become apparent to those skilled in the art. The present technology should, therefore, not be limited by the specific disclosure herein, and can be embodied in other forms not explicitly described here, without departing from the spirit thereof.

We claim:

1. A method of report generation comprising:
receiving a dictation;
selecting a JSON template;
extracting one or more keywords from the dictation;
wherein, when at least one keyword is identified, populating at least one section of the JSON template based on the at least one keyword, and when no keyword is identified, retaining a default version of the JSON template;
assembling a dynamic prompt based on the JSON template and any populated section; and
generating a report based on the dynamic prompt and the dictation.

2. The method of claim 1, wherein extracting the one or more keywords from the dictation comprises:
identifying, in the dictation, one or more words that are present in a lexicon;
isolating the one or more words in context within the dictation; and
classifying the one or more words as the one or more keywords.

3. The method of claim 1, wherein generating the report comprises:
submitting the dynamic prompt to a large language model (LLM); and
generating, by the LLM, the report based on the dynamic prompt and the dictation.

4. The method of claim 3, further comprising:
performing a post-generation reconciliation between (i) the one or more keywords detected in the dictation and (ii) one or more keywords present in the generated report;
wherein, when a keyword detected in the dictation is absent from the generated report, flagging a potential dropped finding; and
wherein, when a keyword appears in the generated report but was not detected in the dictation, signaling a potential hallucinated term.

5. The method of claim 4, further comprising:
when the potential dropped finding is flagged, performing at least one of: inserting a missing keyword into an appropriate section of the report, or prompting a user to confirm placement; and
when the potential hallucinated term is signaled, performing at least one of: removing the potential hallucinated term, qualifying the potential hallucinated term, or prompting the user for confirmation.

6. A system for report generation comprising:
a device for receiving a dictation;
a database with specialized lexicon;
a processor for extracting keywords from the dictation based on the database;
a decision engine for selecting a template;
a processor for constructing a prompt based on the template and any keywords; and
a large language model for generating a report based on the prompt.

7. A method of report generation comprising:
selecting a baseline prompt that includes placeholders corresponding to template sections;
retrieving the JSON template from a template library;
determining, using a lexicon, one or more keywords associated with the dictation;
populating one or more of the placeholders with content derived from the JSON template and the one or more keywords;
modifying the baseline prompt based on one or more rules that are triggered by detection of at least one keyword to generate a final prompt; and
providing the final prompt and the dictation to a large language model (LLM) to generate a report.

8. The method of claim 7, further comprising:
customizing the rules and JSON template for a particular user by at least one of: toggling default phrases, locking or unlocking sections, or selecting a user-specific variant template.

9. The method of claim 8, further comprising presenting a user interface that enables a user to customize the rules and JSON template.

10. The method of claim 9, wherein the user interface enables the user to select a report section and to at least one of (i) edit a default phrase for the report section or (ii) designate the report section as locked against modification by the LLM.

11. The method of claim 9, wherein the user interface enables the user to edit a rule by specifying (i) at least one keyword that triggers the rule and (ii) an action that modifies a corresponding section of the final prompt.

12. The method of claim 9, further comprising enforcing role-based permissions such that an administrator is permitted to edit the JSON template or rules and a non-administrator is restricted to selecting among pre-approved customizations.

13. The method of claim 9, further comprising storing, for each customization, at least one of a version identifier, an effective date, or an audit record identifying a user associated with the customization.

14. The method of claim 9, further comprising:
generating a preview report that shows how the report will appear based on the customization of the rules and JSON template.
